(19) United States
(12) Patent Application Publication
(10) Pub. No.: US 2004/0147975 A1
(43) Pub. Date: Jul. 29, 2004

(54) Functional electrical therapy system (fets)

(76) Inventors: Dejan P. Popovic, Belgrade (YU); Thomas Sinkjaer, Gistrup (DK)

Correspondence Address: Pillsbury Winthrop, Intellectual Property Group, 1600 Tysons Boulevard, McLean, VA, 22102, US

(21) Appl. No.: 10/475,300

(22) PCT Filed: Apr. 23, 2002
(86) PCT No.: PCT/EP02/04466

(30) Foreign Application Priority Data
Apr. 24, 2001 (DK) 2001/00650

(51) Int. Cl. A61N 1/18

(52) U.S. Cl. 607/48

(57) ABSTRACT

The Functional Electrical Therapy System (FETS) relates to an apparatus and the method of its application for recovery of upper limb functioning in humans with sensory-motor lesion in the central nervous system having impact to reaching and grasping. The recovery is contributed by 1) the electrical stimulation of efferent nerves that augment and/or generate missing functions of the upper limb, 2) electrically induced strong input to the central nervous system via afferent nerves, 3) intensive exercise by the same paretic upper limb, and 4) increased awareness of being able to accomplish the task. The methods comprise the use of the novel apparatus that provides missing or diminished functions of the upper limb during intensive daily exercise of performing typical daily activities that requires these functions. The FETS consists of a multi-channel stimulator, set of surface electrodes, and the novel controller that mimics spatial and timing synergies of muscle activation found in able-bodied subjects when performing functional tasks.

The present invention relates to a method and to apparatus for improving the physical ability of persons having disabled upper extremities due to sensory-motor impairment of the central nervous system.

The motor functions of an upper limb that is paretic or paralysed after stroke can be improved by intensive exercise and augmented feedback [Sunderland et al., 1992], or Constraint Induced Movement Therapy [Kunkel et al., 1999; Miltner et al., 1999, Taub et al., 1999]. The use of robots can also enhance the motor functioning [Aisen et al., 1997; Volpe et al., 2000]. Electrical stimulation of peripheral sensory-motor systems works as a therapy, although there are no conclusive directions which technique works the best for a given indication [Kraft et al., 1992; Glanz et al., 1996; Hummelsheim et al., 1997; Feys et al., 1998; Chae et al., 1998; Francisco et al., 1998; Dimitrijevic and Soroker, 1994; Dimitrijevic et al., 1996; Sonde et al., 1998, 2000]. Electrotherapy was introduced by Liberson et al. [1961]; they used a peroneal stimulator to provide contraction of the dorsiflexor muscles based on the trigger signal that came from the heel switch in order to reduce the foot-drop.

In an early use of electrotherapy in upper limbs Merletti et al. [1975] applied a two or three channel electrical stimulator to augment elbow extension and fingers/wrist extension. The device used proportional control, and it allowed independent control of each of the stimulation channels. The patients were hemiplegic. With stimulation, they were able to move an object from one location to another, which they could not do without the stimulation. The conclusions from this small clinical trial were that an improvement was obtained in elbow extension without stimulation over a period of three months, but not of hand opening movement. The conclusions from a small clinical trial were that the electrotherapy improved the hand and elbow control with stimulation in all study subjects.

Stimulation systems for functional support of the upper extremities have been developed for spinal cord injury and stroke, both implanted and with electrodes attached to the skin. In many cases, these systems have been developed to provide immediate functional support, although beneficial therapeutic effects, resulting in improved voluntary movement control, have been reported in several studies. More recently, studies have been performed that investigate the therapeutic effect of electrical stimulation primarily. The neurophysiological basis of these therapeutic effects is still insufficiently understood. Therefore, it is unclear how the therapy should be optimized.

There are several stimulation systems that use several surface electrodes, multi-channel stimulator and a pre-programmed sequence of stimulation that can be triggered by a switch, several switches, or signals from a sensor [Nathan, 1993, Ilic et al., 1994; U.S. Pat. No. 5,330,516; Saxena et al., 1995; Prochazka et al., 1997; U.S. Pat. No. 5,562,707; Popovic and Popovic, 1998; Popovic and Popovic, 2001; Fisekovic and Popovic, 2001]. There are several stimulation systems with implantable electrodes [Hoshimiya et al., 1989; Smith et al., 1987], or fully implantable systems [Smith et al., 1996; U.S. Pat. No. 5,167,229] that use a pre-programmed stimulation that is controlled by a switch or sensory signal in an open-loop or closed-loop control scheme. These stimulators have been designed to operate in humans with chronic paralysis or paresis caused by a cerebro-vascular accident or spinal cord injury (e.g., Popovic et al., 1998; Popovic et al., [1999]. All of these stimulators use the well-known principles described basically in the work of Liberson et al., [1961] and Rebersek and Vodovnik [1973]. Most of the stimulation systems comprise the flexibility to be programmed for various applications other than the one that they are originally designed for; however, the best stimulation protocol is unknown thus the change of the stimulation protocol is not straight forward.

In a first aspect, the present invention provides a method for integrating electrical stimulation and upper extremity exercise in order to improve the physical ability of persons having a disabled upper extremity due to sensory-motor impairment of the central nervous system, said method comprising the steps:

imposing from the apparatus functional electrical stimulation of neural pathways based on a voluntary command using a stimulation pattern that mimics natural flow of neural activities to the impaired upper extremity,

generating the missing components of a functional movement in parallel with the voluntary exercising of the same functional movement based on the said patterned stimulation of the efferent neural pathways time-synchronized with volitional movement,

enhancing the afferent input by the said patterned electrical stimulation in time synchrony with the biological afferent activity caused by the functional movement of the limb.

Most of the previously described techniques considered only to a peripheral degree the manner in which the different treatments benefit from the today very well accepted effects of neural plasticity. Namely, the human brain has a power to change its structure and function in order to optimize the performance of frequently performed activities. The brain learns from its own actions. The afferent sensory information, giving feedback about the consequences of voluntarily controlled actions, is essential for this learning process. It is the way young children learn to move in a functional manner, or professional train for competition. There is increasing evidence that it is also the way adult persons relearn their motor activities after a stroke.

Therapy for motor relearning in persons with paresis caused by stroke should support this process of relearning optimally by functionally assisting the user to perform intended activities, which they may only be able to perform poorly or not without assistance. The sensory feedback associated with the performance of the activities will assist the relearning process of the brain. This functional sensory feedback should be maximized both in intensity and duration and should not be limited to short clinical training sessions.

Functional Electrical Therapy (PET) in accordance with this invention is the ideal modality for this activity dependent neurorehabilitation. FET is (in preferred aspects) a non-invasive neuromuscular stimulation modality that assists motor activities intended by the user, while activating sensory channels that provide maximal afferent inflow to the brain, consistent with the performance of the activities, thus training the neuromuscular system to perform the function independently. FET trains function in contrast to conventional electrical therapy. The FET approach promotes functional reorganization of the brain and, consequently, functional relearning. During the therapy period, a FET system can be provided to each stroke person for use during his or her daily activities, thus resulting in intensive training. Recent studies suggest that FET is a promising modality for promoting the relearning of reaching and grasping in paretic persons [Popovic et al., 2002].

Functional Electrical Therapy (FET) enables a paretic user to perform functional activities during a period when he-she is not yet able to perform these functions self-sustained, thus promoting normal functioning. The functional sensory information thus generated is hypothesized to result in intensive functional brain training of the activities performed. The functional support and maximal sensory functional feedback to the spinal cord and the brain make FET very different and, preliminary data indicate, superior to alternative forms of traditional therapies (neuromuscular facilitation, therapeutic neuromuscular stimulation, below the motor threshold stimulation, intensive exercise, etc.).

Although general theories exist regarding the neurophysiological basis of neurorehabilitation through Functional Electrical Therapy, limited actual knowledge is available about the mechanisms.

Conceptually, the following processes are believed to contribute to the training of the neural and muscular systems when applying Functional Electrical Therapy:

Arm muscles are activated in a functional manner through the stimulation of the peripheral motor nerves. In this way, functional activities, which may not be feasible without support, can be performed. Furthermore, the stimulation results in strengthening of the activated paretic muscles.

The body's sensory system generates sensory signals providing information about the performance of functional activities.

Sensory signals are furthermore generated directly by the excitation of sensory nerve fibers in the stimulated peripheral motor nerves.

The sensory information generated by the body's sensory system and through direct nerve stimulation influences the neural networks in the spinal cord, with impact on the reflexive system, possibly reducing hyper-reflexia and functionally modulating reflexive pathways.

This sensory information furthermore influences the structure and function at the level of the brain, potentially improving volitional motor control performance by learning.

Thus, in accordance with the method of the invention, patients having a deficit in the brain, typically of the type induced by a stroke, rather than in the efferent nerves of the spinal cord or lower, benefit from FETS. By a ‘functional movement’, we mean a desired, previously determined movement, such as opening the hand, engaging an object and closing the hand to grip the object.

In accordance with the method, the patient wills the functional movement to occur and attempts to make the movement by voluntary action of the relevant muscles for performing the movement in the healthy person, at the same time that the apparatus produces the movement by a pre-programmed electrical stimulation of the muscles. Thus, the patterned stimulation of the muscles is synchronised with volitional movement or volition of movement (whether or not the patient is capable of any relevant movement without the stimulation).

The electrical signals are perceived by the patient, which provides an enhanced afferent input in synchrony with biological afferent activity, i.e. exteroceptive signals as well as proprioceptive signals.

The Functional Electrical Therapy System (FETS) comprises an apparatus and the method of its application for recovery of sensory-motor systems in humans central nervous system injury with impact to reaching and grasping that combine the both techniques: electrical stimulation and exercise. An important preferred feature of the FETS is the control that allows that the electrically elicited activity of hand and arm of a person with sensory-motor deficit is synchronised with the extensive exercise; thereby affecting all available resources and leading to the fast and efficient rehabilitation of function [Popovic et al., 2001]. The FETS is designed for short-term therapeutic application; it is not developed for chronic, every day orthotic use alike Freehand® [Smith et al., 1998] or Handmaster NMS-1® systems [Nathan, 1993].

In a preferred method according to the invention the electrical stimulation is provided from the stimulator at a number of the bodily locations by means of surface electrodes. However, the use of implanted electrodes is not excluded.

Preferably, the efferent pathways are pathways from the spinal cord of the disabled person to a muscle in the arm or hand of the disabled person.

A control algorithm is preferably incorporated into the controller, said control algorithm mimicking the natural sequence of activation of muscles responsible for functional movements. The mimicking may provide stimulation comprising triphasic activity of agonist muscles and antagonist muscles and controlling the level of co-contraction of agonists and antagonists.

A voluntary movement in the same upper limb may be used as a source to drive in an open-loop control scheme the activation of more distal parts of the arm. Such movement may be the moving forward of the elbow in a reaching movement.

The afferent pathways may be pathways from the sensory system of the body of the disabled person to the spinal cord of the person.

The steps may be repeated so that the electrical stimulation for a prolonged time provides a strong afferent input that is correlated with the propriceptive and exteroceptive inputs caused by the voluntary movement and by contacts with the environment.

The invention provides in a second aspect apparatus for use in the integrated electrical stimulation and upper extremity exercise in order to improve the physical ability of the upper limb of a disabled person, said apparatus comprising a stimulator for providing electrical signal outputs via a number of stimulation channels, and a number of electrodes for placement in physical contact with the body of the disabled person and means for connecting said electrodes to said stimulator, and a controller providing instructions to said stimulator and implementing a control algorithm that causes the stimulator to provide via said channels stimulation patterns of said electrical signals which induce muscle movements in use which mimic the timing and modulation of muscles typically active in able-bodied humans that are available for surface activation with electrical stimulation in persons with impairment, said signals including non-simultaneous peaks of activation of agonist and antagonist muscles during a single direction of movement (triphasic pattern) and appropriate coactivation of agonist and antagonist muscles needed for a desired functional movement.

Said apparatus may be suitable for use in performing the method of the first aspect of the invention. Suitably, the apparatus is suitable for producing manipulation and grasping of objects with an impaired hand.

The controller may communicate with a number of stimulators, said stimulators comprising lead communication, alternatively or supplementary wireless communication channels between the controller and stimulation points.

The apparatus may be intended for stimulating and controlling fingers, thumb, and forearm of the disabled person, and the number of stimulator channels may comprise at least one channel for the finger flexors, at least one channel for the finger extensors, at least one channel for thumb extension/adduction, and at least one channel for thumb opposition/flexion.

The apparatus may comprise a shoulder angle sensor for determining the angle or rate of change of the angle between the upper arm and a reference line, wherein said controller is adapted to initiate and to regulate a said control algorithm inducing a sequence of reaching, grasping, moving, holding, returning and releasing an object in response to measurement or calculation of said rate of change based upon the output of said shoulder sensor.

The invention further includes the use of the apparatus for improving the physical ability of persons having disabled limbs due to sensory-motor impairment in the brain of the person caused by a lesion of the central nervous system of the person, where the improvement relates to long-term changes of ability to perform movement without the stimulation after the treatment.

Also, the invention includes the use of apparatus for temporary therapeutic applications in persons with sensory motor deficit of an upper limb within the intensive physical exercise, said therapeutic application augmenting and/or generating the missing movement of the hand/arm.

The invention will be further described and illustrated with reference to the accompanying drawings in which:

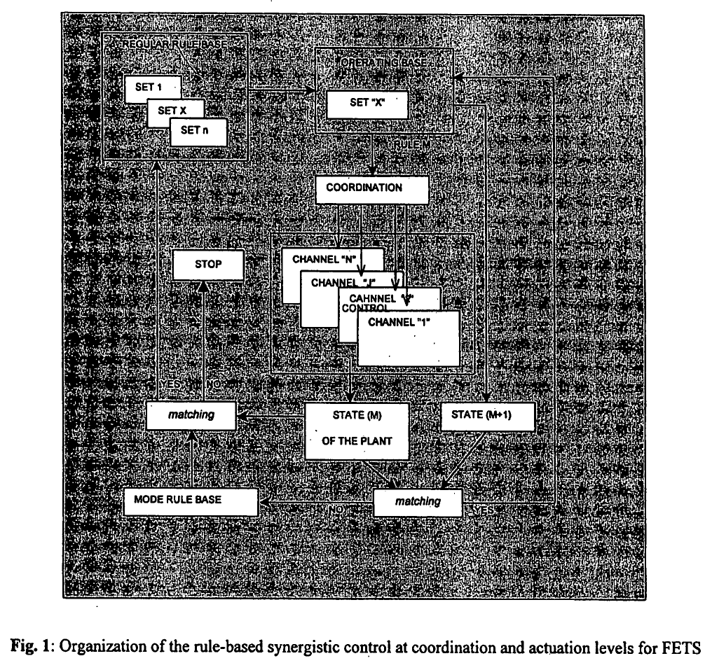

FIG. 1 shows the organisation of a rule based synergistic control for a preferred embodiment of the invention at coordination and actuation levels for FETS;

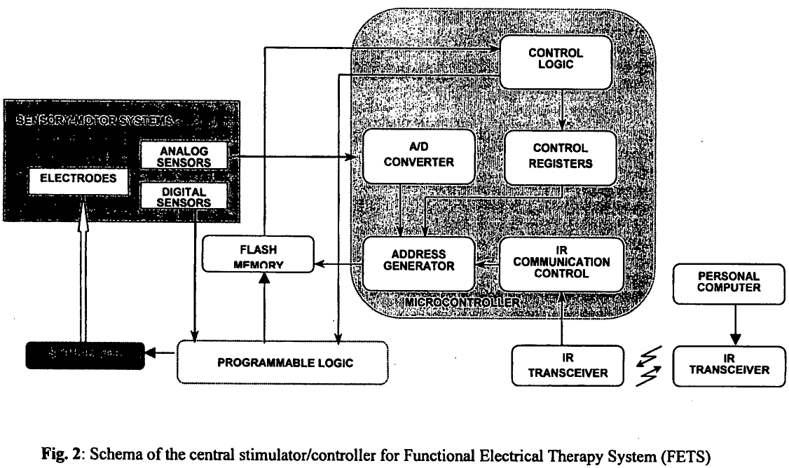

FIG. 2 shows a schematic of the central stimulator/controller of the described preferred embodiment;

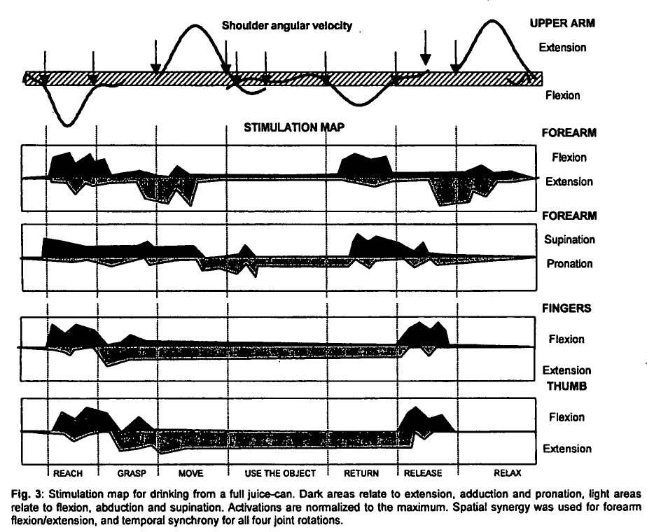

FIG. 3 shows a stimulation map indicating the timing of electrical stimulation to muscles involved in a drinking movement; and

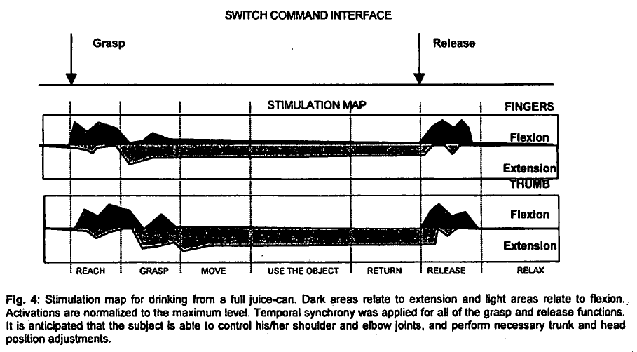

FIG. 4 shows a similar stimulation map for a modified embodiment providing fewer channels for electrode stimulation and provision for input from a shoulder angle reading sensor.

A Functional Electrical Therapy Apparatus comprises the following 1) a multi-channel stimulator that is capable of activating paralyzed innervated muscles when applied with surface electrodes, 2) a controller that is capable of generating command signals that would fit into the biological control of movement. 3) electrodes, 4) sensors, and 5) switches.

1) A multi-channel stimulator that allows stimulation of motor nerves can be used for that providing that it can be controlled in sense of frequency of stimulation, pulse duration, and pulse amplitudes.

The therapeutic stimulation uses pulses that are about 200 microseconds long, have amplitude in the range of 0 to 50 mA, and the frequency of 50 Hz. These parameters have been selected in order to minimize the pain, yet decrease the development of fatigue. The therapeutic stimulation is not meant for static applications (e.g., long lasting grasps), yet for the dynamic behaviors (e.g., move, grasp, release during functional use of objects typically needed in daily life).

Communication link to the controller allows that this stimulator generates specific bursts of activities that could generate various movements. This link allows control of pulse duration and stimulation frequency on each of the channels independently. This means that each channel can be turned at any time at any intensity depending on the control signals delivered through the said link.

2) FETS controller uses a coordination model of movement that has been described in literature dealing with motor control [Popovic, 2002].

The coordination model of reaching and manipulation hypothesizes that there is a high connectivity between individual joint movement that is, muscle activation during functional movements. The connectivity is being developed through a skill acquisition procedure, mostly in childhood. This connectivity allows that a single decision at the brain level to perform a function is appropriately distributed to end organs. This coordination is achieved by the hierarchical and parallel organization of the CNS, yet is can be assessed and presented in different phase spaces (e.g., force space, velocity space extrinsic or intrinsic coordinates). Control of reaching and grasping was found independent (reaching and grasping synergies) [Jeannerod, 1981], but coordinated through a hierarchically higher control mechanism that ensures temporal alignment of reaching and grasping. Dissociation in the control of reaching and grasping was confirmed in comprehensive studies [Gentilucci et al, 1997]. Neurophysiological findings support functional separation of reaching and grasping [Van Kan et al, 1994]. Decoupling between the reaching and grasping controls is suitable for life-like control yet temporal synchrony between the reaching/grasping/releasing movements stays instrumental.

Conventional control techniques developed for machine and robots are not suitable for the multi-joint, multi-actuator biological systems such as the arm/hand complex [Tomovic et al., 1995; Potkonjak et al, 2001] mostly because external control must be integrated into the preserved sensory-motor mechanisms. Incorporating preserved natural features and generating artificial life-like control requires the use of heuristic tools and structuring control in a hybrid hierarchical fashion [Tomovic et al, 1995].

A coordination model of control is shown in FIG. 1; it comprises three automatic, and one biological control level [Popovic, 2002]. The highest control levels (decision and planning) allow the user of a neuroprosthesis to select an action, and the modality of operation, i.e. to provide sufficient detail to specify where, how, and what the movement should accomplish. The role of the second automatic level is to coordinate simple synergies between joints and it could use a discrete rule-base control. A rule-base controller operates as a sampled-data feedback system, and its role is to transfer commands to the lower level and process the feedback information. A rule-base controller implements the state model of movement that is cloned by a heuristic of machine classification using data acquired in able-bodied humans; thereby mimicking life-like movement.

The rules need to be determined with sufficient generality to allow the application over a large population with minimum limitations. The lower coordination is implementing synergies at the joint level, i.e., machine determined mappings between the state variables (e.g., arm segment angular velocities) [Popovic and Popovic, 1994, 2001]. The lowest, actuator level deals with execution, that is, for example, patterns of functional electrical stimulation of muscle groups being responsible for the flexion or the extension of a single joint, or biarticular muscles contributing to the movements of the neighboring joints. The actuator level implements open-loop model-based control than includes individual biomechanical and neuromuscular features of the eventual user.

The FETS deals with two motor system properties of manipulation and grasp: 1) Temporal synchrony; and 2) Reaching synergies.

1. Temporal synchrony. The execution of manipulation and grasp functional task is coordinated through the following phases: 1) the hand transport to the object post (positioning or reaching), hand orientation and opening (prehension), hand closing (grasping of the object); 2) using the object (the actual task); 3) returning the object and releasing it at the object post; and 4) finally returning the hand to the initial position.

2. Spatial synergies. The term spatial synergy means acting together. This can be linked to low-level neural elements (reflex arc), only one step away from the muscle [Sherrington, 1947] or alternatively, at higher-level neural processes i.e. functional [Bernstein, 1967]. The synergy can be anticipated at the level of movement initiations, that is activation of muscles, or at the level of movement performance (execution), that is individual joint rotations [Latash et al, 1999]. Spatial synergy is a rule that establishes a relation among central control variables to individual joints of a limb resulting to a relation among performance variables. Existence of synergies is expected to shift control of multi-joint movements to a higher level, as compared to control of individual joints, and to decrease the number of central variables manipulated by CNS.

There is no other therapeutic system that uses this mode of control. Control algorithm provides alternating stimulation of agonist and antagonist muscles in the order typically found in able-bodied subjects. Control algorithm comprises phases of cocontraction and typical triphasic activity of muscles.

Thus, it can be seen in FIGS. 3 and 4 that during a phase of the desired complex movement in which the movement of a component of the limb, such as the fingers, is in a single direction, e.g. opening of the fingers, the stimulation of the muscles involves peaks of stimulation of agonist and antagonist muscles which follow one another. This is further described below.

The tested controller (FIG. 2) is powerful; it is capable of regulating up to 8 bipolar, mutually independent stimulation channels. The outputs from the controller are logical signals: Low and High. The interpulse interval, reciprocal of the frequency of stimulation, is the time between two subsequent transitions from Low to High; it can be selected to be between 10 ms and 2,55 seconds in increments of 10 ms. The pulse duration is equal to the period of a single “High” state; it can be programmed to be between 10 s and 1270 s in increments of 10 s. The increase and decrease rates of pulse duration are programmable for each of the channels independently. The amount of charge delivered to the motoneurons depends directly on the pulse amplitude, which is set at the stimulator output stage manually. The power generator and output stages anticipated for FES system are a modified version of the stimulator developed by Ilic et al. [1994].

The controller operates in a sensory-driven mode, data from analog sensors are fed to an 8-bit A/D converter (a component of the micro-controller), and/or digital signals from digital sensors go directly to a port on the micro-controller. The controller supports up to a maximum of 6 digital and 8 analog sensors. The sensory output signals must be processed to be between 0V (Low) and 5V (High). Five fully retriggerable timers are integrated in the controller in order to enable the use of a time series of sensory data and special commands. The timers are designed for the range between 0.05 to 50 seconds, with an increment of 0.05 seconds. In order to increase the control capacity, five internal control signals can be generated and stored in the memory of the micro-controller. These control signals define the state of the stimulator and allow multi-modal operation (i.e. different patterns can be activated for the same sensory input). The timers and control signal are important specifically for the application of a hierarchical hybrid rule-based control (Popovic and Sinkjaer, 2000).

The controller comprises the following: a micro-controller, two programmable logic arrays, a flash memory, an infrared (IR) communication port, and a time base generator. The PIC16F877-20I/PT micro-controller is the core of the device (FIG. 2). It co-ordinates all other sections and executes the algorithms. The controller digitises input signals from analog sensors and/or receives digital signals, generates part of the address for access to the flash memory, and forwards the appropriate data to the programmable logic arrays.

Two programmable gate arrays (PGA) perform logic operations. The basic tasks for PGA chips are to generate pulses with defined duration on the basis of information received from the micro-controller. The PGA chips also deal with the logic for the bus control, access to the peripherals and memory modules, and they serve as the interface for the digital sensors.

Intel's flash memory DA28F016SV (capacity 2 MB) was selected based on the estimated size of the programs. The flash memory was preferred to a ROM memory because of the needs to tune and change the programs on site. The process of reading flash memory is the same as reading SRAM with respect speed and access, yet the content remains in the memory after the system is switched off.

The controller and PC communicate via an IR transceiver [Fisekovic and Popovic, 2001]. This cordless communication is only for transmission of the control algorithm (generated at a PC computer) to the stimulator. The IR communication system comprises an infrared transceiver (MINI SIR, Novalog), and the pulse shaping circuit (IC transceiver driver, TOIM3232, Temic). At the PC side there is a MAX232 (Maxim) level conversion chip between the serial port (RS232 standard: ±12V), and the transceiver drivers (5V). The IR transceiver MINI SIR and the circuit TOIM3232 are compatible with IrDA association standards.

In order to simplify the pulse generation a timer (LMC555) is configured for operation as a system basic rhythm clock (self-oscillating flip-flop) at the frequency of 100 kHz, ensuring the minimum pulse duration of 10 μs.

The operation principle of the controller is the following: the data from analog sensors are fed to the microcontroller inputs, and transformed to a digital form. Based on these values, the timer states, and the control register states, the microcontroller generates part of the address for access to the flash memory. This part of the address consists of 16 lower bits, out of a total of 22 bits needed for the access to the memory (21 address bits, and 1 bit for chip selection). The structure of this part of the address is as follows: the six lowest bits define the data from the control algorithm that is currently read, and the channel to which the reading applies. The nine following bits are distributed among A/D converters, timers, and control signals, and the distribution is defined by the stimulator configuration. These nine bits are actually defined by the current state of input and control values of the stimulator, and they determine the action that should be performed by the control algorithm. The sixteenth bit represents chip selection, and it defines whether the next instruction from the micro-controller program is read in the current memory cycle, or whether the flash memory is accessed and data defined by the control algorithm are read. The program generates the remaining six address bits on the basis of the states of digital sensors. A combination of these six bits and the nine bits generated by the micro-controller defines the immediate control data. These data are sent to the programmable logic arrays (frequency and pulse duration). Then, the new timer states and control signals are set. Finally, the pulses generated by the micro-controller are fed to opto-couplers that drive the output stages of the stimulator. Based on the data read, the micro-controller also selects the channels that should be active.

The interface program for entering the control algorithm and its parameters is supported by Windows operating systems with the minimal hardware requirements (PC based computer with a VGA graphics card).

The programmer initialises the set-up, and he/she is prompted with the main menu. The menu enables selecting one of the following six program activities: 1) configuration, 2) algorithm definition, 3) program file, 4) generation of the stimulator code, 5) load and save, and 6) exit, as follows:

1) Configuration. This section of the program is used to define the actual configuration of the stimulator for the selected operation: active A/D channels and their range, timers and control signals that will be used, number of channels that will be used, and unipolar or bipolar regimes. It is desirable to decrease the large number of input signal levels with a smaller set of values [Tomovic et al., 1995]; thus, 2, 4 or 8 levels of analog signals are allowed. In order to minimise effects from the noise recorded by sensors and noise introduced by the A/D conversions, a hysteresis of the sensory signal readings was introduced: an upper and lower threshold defines each sensory level. The configuration section of the program is used to choose how many levels will be used. The software automatically checks for contradictory configurations.

2) Algorithm definition. This section is the main part of the program. It allows the user to define the conditions and appropriate modality of stimulation. The combination defined by the states of the sensors (digital and analog), timer, and control signal states is entered in the definition of the algorithm. The definition of the algorithm opens a mask where the programmer sets the pattern of stimulation. This section allows setting of the minimum, maximum, rise, and fall times of pulses for each channel independently, and it allows the set-up of the interpulse intervals. This programming applies when the stimulator is to be used for therapy and exercise. The algorithm definition is automatically downloaded from the machine learning program for functional movements like walking [Jonic et al., 1999].

3) Program file. This section offers two possibilities. The first is named condition check. By starting this option, the program examines whether there are contradictions in the control algorithm (e.g., more than one pattern of stimulation is defined for the same set of input parameters). If contradictory conditions are detected, the program informs the user about the conditions that are contradictory, and they have to be manually corrected. The second option is program file generating. This option generates a file containing data formatted to suit the micro-controller.

4) Stimulator programming. This section is used for transferring the file from the PC computer to the micro-controller memory.

5) Load and save. This section allows the programmer to save the defined program on the PC computer for later use. It is possible to save the control algorithm and the stimulator configuration, or only the configuration.

6) Exit. This section closes the programming sequence and prompts the programmer to the unsaved information before exiting.

The control algorithm provides a so-called life-like control. The operation of the controller can be classified to sensory-driven or switch-triggered open-loop procedure [Popovic and Popovic, 2001, Tomovic et al., 1995]. The control delivers signals that sequentially activate programmed bursts of electrical pulses to agonist and antagonist muscles of distal arm segments. The cathodes are positioned so to deliver charge to finger flexors, finger extensors, thumb extensor and flexors following the grasping synergy and the timing that mimics the movement seen in healthy subjects when reaching and grasping. The key element is the timed activation of finger and thumb extensors for palmar and lateral grasps that provide opening during the prehension phase, and time activation of thumb and finger flexors during the closing phase of the grasp. The control algorithm incorporates the control of pronation and supination of the forearm, as well as elbow flexion/extension, yet they do not have to be employed if so decided.

The actual change of the pattern (grey areas) is obtained by the change of the pulse duration in real-time. The stronger the stimulation needs to be the longer the pulse should be. The adjusting of the pulse duration vs. the generate movement is individual. The controller of the FETS uses a template type adjusting; patients tune is by adjusting the overall intensity of the pulse intensity, that is, they are setting the level to which all signals are than normalised.

The actual change of the pattern (grey areas) is obtained by the change of the pulse duration in real-time. The stronger the stimulation needs to be the longer the pulse should be. The adjusting of the pulse duration vs. the generate movement is individual. The controller that is part of the FETS uses a template type adjusting, it is not tuned to each patient wit the pulse duration; patients tune is by adjusting the overall intensity of the pulse

The graph presented shows two important elements. There is two couplings: 1) a coupling between the intensity of stimulation and movement in proximal joint at the level of control of reaching (elbow movement, pronation/supination), and 2) timing coupling between the onset of stimulation for opening and closing of the hand coming for the sensor on the proximal muscle, or from a switch if the proximal segments are not instrumented with electrodes or sensors.

The controller is responsible for, as shown in FIGS. 3 and 4, alternating and/or simultaneous, if so required, stimulation of agonist and antagonist muscles.

The control scheme was developed based on detailed analysis of able-bodied persons movement during functional tasks. The subjects were seated in front of a desk and asked to slowly do many manipulation and grasping tasks. The tasks (e.g. drinking from a full juice can and small bottle, handling a video-cassette and CD, writing with a pen, eating finger food) were selected to include the following: 1) palmar, lateral, and precision grasp; 2) different positions within the working space defined with the following attributes: distance and laterality, and 3) various sizes and weights of objects.

The volunteer subjects were asked to repeat for five times each functional task using their dominant arm. Four subjects were left handed, and six right handed. The data was recorded in all subjects during three independent sessions. The order of tasks was randomly changed. Subjects were asked to slowly move their arm while performing a task (drink, eat, write, etc). Subjects were instructed to stop their movement once they accomplish the grasp, at the moment that they start the functional use of the object (e.g., drink, write), and at the time after they released the object at the object post.

Data were collected (three dimensional movement analysis) and off-line processed. The processing provided the tabulated angular velocities that could be automatically analyzed. The task of analysis was to determine the minimal number of unique coupling between joint angular velocities that characterize the task, position, size, or their combination. Two forms of couplings were of interest: the spatial mapping and the time synchrony. The duration of movement duration was normalized for each series of measurements. We used advanced machine learning techniques for determining the couplings between the joint angular velocities, as well as the timings between different events to be controlled.

The second element for designing control was to analyze the stimulation pattern that is required to produce different movement when using surface stimulation. The method for designing stimulation patterns was described in details in Popovic et al. [1994]. The method assumes a series of measurements in the potential user for determining joint and muscle properties. A detailed analysis of joint and muscular properties in many stroke subjects showed that it is possible to define the stimulation pattern with respect the maximum stimulation level, and that it is sufficient to divide the subjects in the group of highly spastic, moderately spastic, and minimally spastic strokes. This allowed the simplified design of FETS control that requires only individual adjustment of the maximum stimulation level (hand controlled potentiometer on the FETS), while the remaining adjustments are done automatically.

3) Set of electrodes being safe for delivering electrical charge for periods of approximately 30 minutes two times a day for a limited interval (e.g. 6 weeks).

These electrodes are daily positioned by the therapist or other trained professional. The electrode system does not require self-mounting. The reusable polymer based electrodes are likely to be the best choice. The size of an electrode has to be individually selected and sized based on the required position on the hand, forearm, or upper arm, the size of the hand and arm, and degree. of atrophy. This system is meant for stroke patients; thus, they are capable of positioning of the electrodes on their own with the good hand once they are trained where to place the electrodes.

The simplest system requires the use of four stimulation channels applied via 5 or 6 electrodes. The cathode for the extension of fingers (opening of the hand) is positioned over the muscle nerve of extensor digitorum communis, and the anode over the bulk of the muscle. The cathode for fingers flexion is positioned over the muscle nerves for flexor digitorum profundus and superficialis, while the anode is positioned over the ulnar and median nerves. The channel responsible for the extension of the thumb is positioned over the muscle nerve of the extensor pollicis longus and the anode over the ulnar and median nerve. Finally the channel that provides thumb closure is positioned over the thenar muscle group.

The more complex system includes four more channels for stimulation that control pronation/supination (cathodes are positioned over the pronator and supinator muscle nerves, and the anodes over the bulk of the muscles. The remaining two channels control the elbow flexion and extension. The electrodes are positioned over the biceps brachii and triceps brachii muscles. The exact positioning must be determined on the individual basis by using specially designed probe that is used as a cathode. The positioning once determined changes very little, yet the individual differences are substantial preventing in having a universal electrode holder or similar interface.

4) Sensors for the system detect the joint angle. The sensors used in this system are goniometers that supply data used to determine the elbow and shoulder flexion/extension angular velocities. The goniometer is not part of this invention, yet its used is.

The role of the sensor is shown in the FIG. 3. The figure shows the operation of eight channels for functional drinking.

The top panel shows the angular velocities measured at the shoulder joint. The angular velocities can be measured directly or estimated in real-time from the joint angle measurements. The joint angular velocities are used for two purposes. When the joint angular velocity crosses the threshold for both positive and negative joint angular velocities (shaded rectangle at the top panel, FIG. 3), then the timing is determined that triggers the required activity (e.g., first flexion starts the prehension and approach phases in all shown muscles, and when this flexion angular velocity becomes small enough the reach phase is over). The joint angular velocity is in parallel used with the predetermined mapping to control in feed-forward manner the pronation/supination and elbow flexion/extension. The first extension that is bigger than the threshold starts the “move” to the functional use phase. The second flexion crossing starts the forearm orientation for accomplishing function. The third flexion angular velocity crossing of the threshold starts the return phase of the hand in order to release the object used. A very short over the threshold extension triggers the mid release phase. Finally the last extension crossing of the threshold starts the relaxation phase.

The functional drinking includes reaching to the object with adequate orientation of the hand, opening of the hand, adjusting the hand for a firm grasp, bringing the object to mouth, rotating the object in order to drink, drinking the object to the object task, releasing the object, returning of the hand to the initial position, and relax state. One can extract any combination of the channels to be used based on the specific needs of the given patient. The reduced number of channels uses the same schema, yet less than eight channels are connected to electrodes.

5) A switch may be provided for initiating operation of FETS. The switch provides the instrumental interface to the user. The switch turns on and off the operation and allows intervention at any time. Such a switch is at a higher level of control than sensors. Any switch that provides instant operation and has the de-bouncing properties can be used within the system.

An example of using only the four channels that control the grasp is presented in FIG. 4 and it is a subset of the channels shown in FIG. 3.

FIG. 4 shows the timing when switch triggers the activity (arrows). A sequence of grasping is divided to the prehension phase (opening of the hand and shaping it correctly for the object size and shape), and grasp where firm contact and safe grasp are accomplished. The grasp phase will last as long as the subject does not trigger the release phase. The pattern of stimulation is not straightforward; it was designed to follow the triphasic pattern of activity that promotes the smooth movement, yet also the enhanced activity of afferent pathways. In parallel, the necessary levels of cocontraction provide smooth and safe graph in addition to ability to fast respond to possible need for changing the posture of the hand.

Thus, it can be seen that upon operation of the switch a sequence of stimulating pulses is sent to each electrode. Looking first at the pulses sent to the finger flexion producing muscles (upper panel, dark grey) it can be seen that the intensity of the pulses (represented by the height of the shaded area above the baseline at any instant) increases rapidly to a peak. Simultaneously with this, pulses sent to the antagonist muscles controlling extension of the fingers (upper panel, light grey area) increase slowly to a peak spaced in time from the agonist stimulation peak. Then the stimulation of the antagonist muscles is sharply reduced, whilst the stimulation of the agonist muscles rises again to a subsequent peak. This alternation of peaks in the stimulation of the agonist and antagonist muscles can be seen to characterise all of the phases of movement.

Thus in summary, a preferred aspect of the invention is the provision of a Functional Electrical Therapy System (FETS) which relates to an apparatus and the method of its application for recovery of upper limb functioning in humans with sensory-motor lesion in the central nervous system having impact to reaching and grasping. The recovery is contributed by 1) the electrical stimulation of efferent nerves that augment and/or generate missing functions of the upper limb, 2) electrically induced strong input to the central nervous system via afferent nerves, 3) intensive exercise by the same paretic upper limb, and 4) increased awareness of being able to accomplish the task. The methods comprise the use of the novel apparatus that provides missing or diminished functions of the upper limb during intensive daily exercise of performing typical daily activities that requires these functions. The FETS consists of a multi-channel stimulator, set of surface electrodes, and the novel controller that mimics spatial and timing synergies of muscle activation found in able-bodied subjects. when performing functional tasks.

### References

1. Aisen M L, Krebs H I, Hogan N. McDowell F, Volpe B T. The effect of robot-assisted therapy and rehabilitative training on motor recovery following stroke. Arch Neurol 54:4 443-6, 1997.

2. Bernstein N A. The coordination and regulation of movements. London: Pergamon Press, 1967.

3. Chae J, Bethoux F, Bohine T, Dobos L, Davis T, Friedl A. Neuromuscular stimulation for upper extremity motor and functional recovery in acute hemiplegia. Stroke 29:5 975-9, 1998.

4. Dimitrijevic M M, Soroker N. Mesh-glove 2: Modulation of residual upper limb motor control after stroke with whole-hand electric stimulation. Scand J Rehabil Med 26:4 187-90, 1994.

5. Dimitrijevic M M, Stokic D S, Wawro A W, Wun C C. Modification of motor control of wrist extension by mesh-glove electrical afferent stimulation in stroke patients. Arch Phys Med Rehabil 77:3 252-8, 1996.

6. Feys H M, De Weerdt W J, Selz B E, Cox Steck G A, Spichiger R, Vereeck L E, et al. Functional magnetic resonance imaging of the human motor cortex before and after whole-hand afferent electrical stimulation. Scand J Rehabil Med 31:3 165-73, 1999.

7. Fisekovic N, Popovic D B. New Controller for Functional Electrical Stimulation Systems, Med Eng Phys, 23:391-399, 2001.

8. Francisco G, Chae J, Chawla H, Kirshblum S, Zorowitz R, Lewis G, Pang S. Electromyogram-triggered neuromuscular stimulation for improving the arm function of acute stroke survivors: a randomised pilot study. Arch Phys Med Rehabil 79:5 570-5, 1998.

9. Gentilucci M, Negrotti A, Gangitano M. Planning an action. Exp Brain Res 115:116-128, 1997.

10. Glanz M, Klawansky S, Stason W, Berkey C, Chalmers T C. Functional electrostimulation in poststroke rehabilitation: a meta-analysis of the randomised controlled trials. Arch Phys Med Rehabil 77:6 549-53, 1996.

11. Hoshimiya N, Naito N, Yajima M, Handa Y. A Multichannel FES System for the Restoration of Motor Functions in High Spinal Cord Injury Patients: A Respiration-Controlled System for Multijoint Upper Extremity. IEEE Trans Biomed Eng BME-36:754-760, 1989.

12. Hummelsheim H, Maier-Loth M L, Eickhof C. The functional value of electrical muscle stimulation for the rehabilitation of the hand in stroke patients. Scand J Rehabil Med 29:1 3-10, 1997.

13. Ilic M, Vasiljevic D, Popovic D B. A programmable electronic stimulator for FES systems. IEEE Trans Rehabil Eng TRE-2:234-239, 1994.

14. Jeannerod M Intersegmental coordination during reaching at natural visual objects. In: Long J, Baddeley A (eds.). Lawrence Erlbaum Associates, Hillsdale, N.J., 1981.

15. Jonic S, Jankovic T, Gajic V, Popovic D B. Three machine learning techniques for automatic determination of rules to control locomotion. IEEE Trans Biomed Eng BME-46(3):300-310, 1999.

16. Kraft G H, Fitts S S, Hammond M C. Techniques to improve function of the arm and hand in chronic hemiplegia. Arch Phys Med Rehabil 73:3 220-7, 1992.

17. Kunkel A, Kopp B, Müller G, Villringer K, Villringer A, Taub E, Flor H. Constraint-induced movement therapy for motor recovery in chronic stroke patients. Arch Phys Med Rehabil 80:6 624-8, 1999.

18. Latash M L, Alexander A S, Zatsiorsky V M. The basis of a simple synergy: reconstruction of joint equilibrium trajectories during unrestrained arm movements. Hum Movt Sci 18:3-30, 1999.

19. Liberson W F, Holmquest H J, Scott D, Dow A. Functional electrotherapy: stimulation of the peroneal nerve synchronized with the swing phase of the gait in hemiplegic patients. Arch Phys Med Rehab 42:101-105, 1961.

20. Merletti R, Acimovic R, Grobelnik S, Cvilak G. Electrophysiological orthosis for the upper extremity in hemiplegia: feasibility study. Arch Phys Med Rehabil 56:12 507-513, 1975.

21. Miltner W H, Bauder H, Sommer M, Dettmers C, Taub E. Effects of constraint-induced movement therapy on patients with chronic motor deficits after stroke: a replication. Stroke 30:3 586-92, 1999.

22. Nathan, R H. Control strategies in FNS systems for the upper extremities. CRC Press Crit Rev Biomed Eng, 21(6):485-568, 1993.

23. Popovic D B, Popovic M B, Stojanovic A, Pjanovic A, Radosavljevic S, Vulovic D. Clinical Evaluation of the Belgrade grasping system, Proc 5^{th} Vienna Workshop on FES, Vienna, 247-250, 1998.

24. Popovic D B, Popovic M B. Belgrade grasping system. J Electronics 2:21-28, Banja Luka, 1998.

25. Popovic D B, Sinkjar T. Control of movement for the physically disabled, Springer, 2000.

26. Popovic D B, Stojanovic A, Pjanovic A, Radosavljevic S, Popovic M B, Jovic S, Vulovic D. Clinical evaluation of the bionic glove. Arch Phys Med Rehabil 80:299-304, 1999.

27. Popovic M B, Jovanovic N, Schwirtlich L. Identification of parameters for control of assistive systems. J Aut Control, Belgrade 4:31-45, 1994.

28. Popovic M B, Popovic D B. A new approach to reaching control for tetraplegic subjects. J Electromyog and Kinesiol 4:242-253, 1994.

29. Popovic M B, Control of upper-extremity neuroprosthesis, Med Eng Phys, 2002 (submitted).

30. Popovic M B, Popovic D B, Sinkjaer T, Stefanovic A, Schwirtlich L. Restitution of reaching and grasping promoted by functional electrical therapy. Proc 6^{th} Vienna Workshop on FES, Vienna, September, 210-213, 2001.

31. Popovic M B, Popovic D B. Cloning Biological Synergies Improves Control of Elbow Neuroprosthesis. IEEE EMB Magazine, 24(1):74-81, 2001.

32. Popovic, D B, Popovic M B, Sinkjaer T. Neurorehabilitation of Upper Extremities in Humans with Sensory-Motor Impairment. Neuromod, 5(1):54-67, 2002

33. Potkonjak V, Kostic D, Tzafestas S, Popovic M B, Lazarevic M and Djordjevic G. Human-like behavior of robot arms: general considerations and the handwriting task-Part II: the robot arm in handwriting. Rob Comp Integr Manuf 17:317-327, 2001.

34. Prochazka A, Gauthier M, Wieler M, Kenwell Z. The Bionic glove: an electrical stimulator garment that provides controlled grasp and hand opening in quadriplegia. Arch Phys Med Rehabil 78(6):608-614, 1997.

35. Rebersek S, Vodovnik L. roportionally controlled functional electrical stimulation of hand. Arch Phys Med Rehabil 54:378-382, 1973.

36. Saxena S, Nikolic S, Popovic D B, An EMG-controlled grasping system for tetraplegics, J Rehabil Res & Develop, 32:17-24, 1995.

37. Sherrington C S. The integrative action of the nervous system. 2^{nd} ed. New Haven Conn.: Yale University Press, 1947.

38. Smith B T, Peckham P H, Keith M W, Roscoe D D, An externally powered, multichannel, implantable stimulator for versatile control of paralyzed muscle. IEEE Trans Biomed Eng BME-34(7):499-508, 1987.

39. Smith B T, Mulcahey B J, Betz R P. Development of an upper extremity FES system for individuals with C4 tetraplegia. IEEE Trans Rehab Eng TRE-4:264-270, 1996.

40. Smith B T, Tang Z, Johnson M W, Pourmehdi S, Gazdik M M, Buckett J R, Peckham P H. An externally powered multichannel, implantable stimulator-telemeter for control of paralyzed muscles. IEEE Trans Biomed Eng BME-45:463-475, 1998.

41. Sonde L, Gip C, Fernaeus S E, Nilsson C G, Viitanen M. Stimulation with low frequency (1.7 Hz) transcutaneous electric nerve stimulation (low-tens) increases motor function of the post-stroke paretic arm. Scand J Rehabil Med 30:2 95-9, 1998.

42. Sonde L, Kalimo H, Fernaeus S E, Viitanen M. Low TENS treatment on post-stroke paretic arm: a three-year follow-up. Clin Rehabil 14:1 14-9, 2000.

43. Sunderland A, Tinson D J, Bradley E L, Fletcher D, Langton Hewer R, Wade D T. Enhanced physical therapy improves recovery of arm function after stroke. A randomised controlled trial. J Neurol Neurosurg Psychiatry 55:7 530-5, 1992.

44. Taub E, Uswatte G, Pidikiti R. Constraint-Induced Movement Therapy: a new family of techniques with broad application to physical rehabilitation—a clinical review [see comments], J Rehabil Res Dev 36:3 237-51, 1999.

45. Tomovic R, Popovic D B, Stein R. Nonanalytical Methods for Motor Control, World Sci Publ, Singapore, 1995.

46. Van Kan PLE, Horn K M, Gibson A R. The importance of hand use to discharge of interpositus neurones of the monkey. J Physiol (Lond.), 480: 171-190, 1994.

47. Volpe B T, Krebs H I, Hogan N, Edelstein OTR L, Diels C, Aisen M. A novel approach to stroke rehabilitation: robot-elicited sensorimotor stimulation, Neurology 54:10 1938-44, 2000.

1. A method for integrating electrical stimulation and upper extremity exercise in order to improve the physical ability of persons having a disabled upper extremity due to sensory-motor impairment of the central nervous system, said method comprising the steps:
imposing from the apparatus functional electrical stimulation of neural pathways based on a voluntary command using a stimulation pattern that mimics natural flow of neural activities to the impaired upper extremity,
generating the missing components of a functional movement in parallel with the voluntary exercising of the same functional movement based on the said patterned stimulation of the efferent neural pathways time-synchronized with volitional movement,
enhancing the afferent input by the said patterned electrical stimulation in time synchrony with the biological afferent activity caused by the functional movement of the limb.

2. A method according to claim 1, where the electrical stimulation is provided from the stimulator at a number of the bodily locations by means of surface electrodes.

3. A method according to claim 1 or claim 2, where the efferent pathways are pathways from the spinal cord of the disabled person to a muscle in the arm or hand of the disabled person.

4. A method according to claim 1, where a control algorithm is incorporated into the controller, said control algorithm mimicking the natural sequence of activation of muscles responsible for functional movements; mimicking relating to triphasic activity of agonists and antagonists and controlling the level of co-contraction of agonists and antagonists.

5. A method according to any of preceding claims of using a voluntary movement in the same upper limb as a source to drive in an open-loop control scheme the activation of more distal parts of the arm.

6. A method according to any of preceding claims, where the afferent pathways are pathways from the sensory system of the body of the disabled person to the spinal cord of the person.

7. A method according to claim 1 where the steps are repeated so that the electrical stimulation is for a prolonged time providing a strong afferent input that is correlated with the propriceptive and exteroceptive inputs caused by the voluntary movement and contacts with the environment.

8. Apparatus for use in the integrated electrical stimulation and upper extremity exercise in order to improve the physical ability of the upper limb of a disabled person, said apparatus comprising a stimulator for providing electrical signal outputs via a number of stimulation channels, and a number of electrodes for placement in physical contact with the body of the disabled person and means for connecting said electrodes to said stimulator, and a controller providing instructions to said stimulator and implementing a control algorithm that causes the stimulator to provide via said channels stimulation patterns of said electrical signals which induce muscle movements in use which mimic the timing and modulation of muscles typically active in able-bodied humans that are available for surface activation with electrical stimulation in persons with impairment, said signals including non-simultaneous peaks of activation of agonist and antagonist muscles during a single direction of movement (triphasic pattern) and appropriate coactivation of agonist and antagonist muscles needed for a desired functional movement.

9. Apparatus according to claim 8, for producing manipulation and grasping of objects with an impaired hand.

10. Apparatus according to claim 8 or claim 9, where the controller communicates with a number of stimulators, said stimulators comprising lead communication, alternatively or supplementary wireless communication channels between the controller and stimulation points.

11. An apparatus according to any of claims 8 to 10, where the apparatus is intended for stimulating and controlling fingers, thumb, and forearm of the disabled person, and where the number of stimulator channels comprises at least one channel for the finger flexors, at least one channel for the finger extensors, at least one channel for thumb extension/adduction, and at least one channel for thumb opposition/flexion.

12. Apparatus as claimed in any one of claims 8 to 11, further comprising a shoulder angle sensor for determining the angle or rate of change of the angle between the upper arm and a reference line, wherein said controller is adapted to initiate and to regulate a said control algorithm inducing a sequence of reaching, grasping, moving, holding, returning and releasing an object in response to measurement or calculation of said rate of change based upon the output of said shoulder sensor.

13. Use of the apparatus according to any of claims 8-10 for improving the physical ability of persons having disabled limbs due to sensory-motor impairment in the brain of the person caused by a lesion of the central nervous system of the person, where the improvement relates to long-term changes of ability to perform movement without the stimulation after the treatment.

14. Use of apparatus for temporary therapeutic applications in persons with sensory motor deficit of an upper limb within the intensive physical exercise, said therapeutic application augmenting and/or generating the missing movement of the hand/arm.
